(19) United States
(12) Patent Application Publication
(10) Pub. No.: US 2016/0251316 A1
(43) Pub. Date: Sep. 1, 2016

(54) PROCESS FOR THE PREPARATION OF ENZALUTAMIDE

(71) Applicant: SUN PHARMACEUTICAL INDUSTRIES LIMITED, Mumbai, Maharashtra (IN)

(72) Inventors: Ramendra Singh RATHORE, Farrukhabad (IN); Venugopal Venkatarama DURVASULA, Vizianagaram (IN); Amit SHARMA, Panchkula (IN); Ram Chander ARYAN, New Delhi (IN)

(21) Appl. No.: 15/033,093

(22) PCT Filed: Oct. 30, 2014
(86) PCT No.: PCT/IB2014/065716

§ 371 Date: Apr. 28, 2016

(30) Foreign Application Priority Data
Oct. 31, 2013 (IN) 3232/DEL/2013

(51) Int. Cl. C07D 233/86; C07C 237/30; C07C 231/12

(52) U.S. Cl. CPC C07D 233/86; C07C 231/12; C07C 237/30

(57) ABSTRACT

The present invention provides a process for the preparation of enzalutamide.

### FIELD OF THE INVENTION

The present invention provides a process for the preparation of enzalutamide.

### BACKGROUND OF THE INVENTION

Enzalutamide is chemically described as 4-{3[4-cyano-3-(trifluoromethyl)phenyl]-5,5-dimethyl-4-oxo-2-sulfanylideneimidazolidin-1-yl}-2-fluoro-N-methylbenzamide of Formula I.

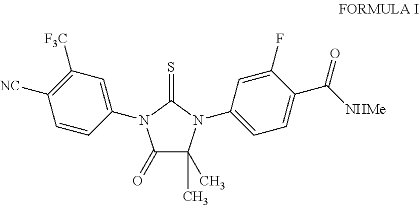 [SMILES: CNC(=O)c1ccc(N2C(=S)N(c3ccc(C#N)c(C(F)(F)F)c3)C(=O)C2(C)C)cc1F]

Processes for the preparation of enzalutamide are described in U.S. Publication Nos. 2007/0004753 and 2007/0254933 and PCT Publication Nos. WO 2007/127010, WO 2006/124118, and WO 2011/106570.

PCT Publication No. WO 2011/106570 discloses that the processes described in U.S. Publication Nos. 2007/0004753 and 2007/0254933 result in a 25% yield of enzalutamide in the final step, which accounts for a 15% overall yield. PCT Publication No. WO 2011/106570 further discloses that the known processes for preparing enzalutamide involve the use of extremely toxic reagents, for example, acetone cyanohydrin.

Acetone cyanohydrin is toxic and therefore its use as a reagent should be avoided for industrial production of a pharmaceutical ingredient. Thus, there is a need in the art to develop a process for the preparation of enzalutamide that avoids the use of acetone cyanohydrin as a reagent.

### SUMMARY OF THE INVENTION

The present invention provides a process for the preparation of enzalutamide that does not involve the use of any toxic reagents and, at the same time, results in a higher yield of enzalutamide.

### DETAILED DESCRIPTION OF THE INVENTION

The term “about”, as used herein, refers to any value which lies within the range defined by a number up to ±10% of the value.

A first aspect of the present invention provides a process for the preparation of enzalutamide of Formula I,

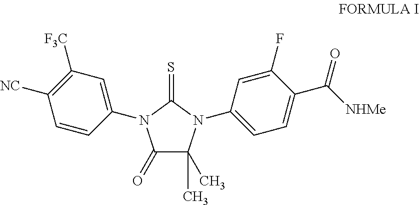 [SMILES: CNC(=O)c1ccc(N2C(=S)N(c3ccc(C#N)c(C(F)(F)F)c3)C(=O)C2(C)C)cc1F]

which comprises:
a) reacting a compound of Formula II

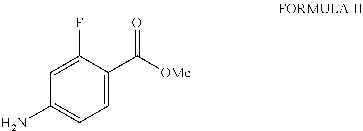 [SMILES: COC(=O)c1ccc(N)cc1F]

with a compound of Formula III

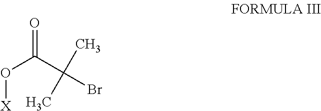

to prepare a compound of Formula IV;

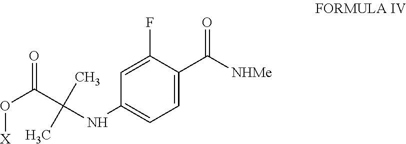

b) reacting the compound of Formula IV obtained in step a) with a compound of Formula V; and

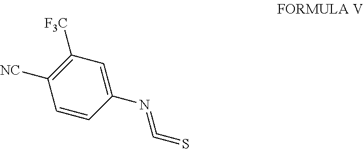 [SMILES: N#Cc1ccc(N=C=S)cc1C(F)(F)F]

c) isolating enzalutamide of Formula I from the reaction mixture of step b) wherein X is methyl, ethyl, or benzyl.

A second aspect of the present invention provides a process for the preparation of a compound of Formula IV,

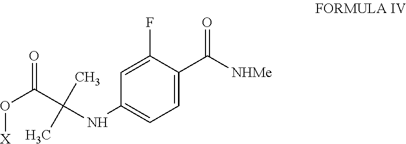

comprising reacting a compound of Formula II

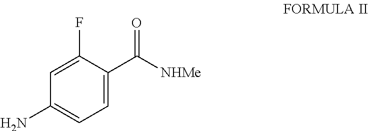 [SMILES: CNC(=O)c1ccc(N)cc1F]

with a compound of Formula III,

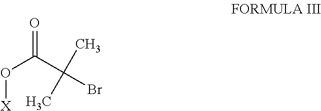

wherein X is methyl, ethyl, or benzyl.

A third aspect of the present invention provides a compound of Formula IV, wherein X is methyl, ethyl, or benzyl.

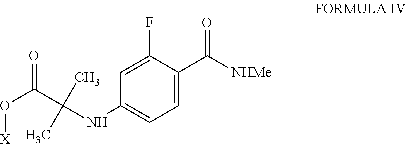

A fourth aspect of present invention provides the use of a compound of Formula II or a compound of Formula IV for the preparation of enzalutamide.

The compound of Formula II can be prepared by reducing a compound of Formula VI.

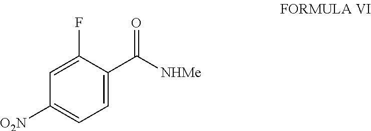 [SMILES: CNC(=O)c1ccc([N+](=O)[O-])cc1F]

The reduction of the compound of Formula VI is performed in the presence of a reducing agent and a solvent. Examples of reducing agents include hydrogen gas in the presence of palladium/carbon, palladium hydroxide/carbon, or platinum dioxide. Preferably, the reducing agent is hydrogen gas in the presence of palladium/carbon. The solvent is selected from the group comprising alcohols, halogenated hydrocarbons, esters, hydrocarbons, ethers, and mixtures thereof. Examples of alcohol solvents include methanol, ethanol, and n-butanol. An example of a halogenated hydrocarbon is dichloromethane. Examples of ether solvents include tetrahydrofuran and diisopropyl ether. Examples of ester solvents include ethyl acetate, butyl acetate, and isopropyl acetate. Examples of hydrocarbon solvents include hexane and heptane. Preferably, the solvent used for the reduction of a compound of Formula VI is methanol.

The compound of Formula VI, which is used for the preparation of the compound of Formula II, can be obtained by reacting a compound of Formula VII

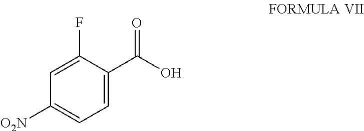 [SMILES: O=C(O)c1ccc([N+](=O)[O-])cc1F]

with methylamine hydrochloride in the presence of a coupling agent and a solvent. Examples of coupling agents include hydroxybenzotriazole, 1-ethyl-3-(3-dimethylaminopropyl)carbodiimide hydrochloride, dicyclohexyl carbodiimide, O-(benzotriazol-1-yl)-N,N,N′,N′-tetramethyluronium tetrafluoroborate (TBTU), and O-(benzotriazol-1-yl)-N,N,N′,N′-tetramethyluronium hexafluorophosphate (HBTU). The preferred coupling agent is hydroxybenzotriazole. The solvent is selected from the group consisting of alcohols, halogenated hydrocarbons, esters, hydrocarbons, ethers, and mixtures thereof. Examples of alcohol solvents include methanol, ethanol, and n-butanol. An example of a halogenated hydrocarbon is dichloromethane. Examples of ether solvents include tetrahydrofuran and diisopropyl ether. Examples of ester solvents include ethyl acetate, butyl acetate, and isopropyl acetate. Examples of hydrocarbon solvents include hexane and heptane. The preferred solvent is dichloromethane.

The compound of Formula VII can be prepared by any method known in the art, for example, PCT Publication Nos. WO 2007/127010 and WO 2006/124118.

In an embodiment of the present invention, the reaction of a compound of Formula II with a compound of Formula III is carried out in the presence of a base and a solvent.

The base can be an organic or an inorganic base. Examples of organic bases include ethyl amine, diisopropyl amine, diisopropyl ethyl amine, and mixtures thereof. Examples of inorganic bases include hydroxides, carbonates, and bicarbonates of an alkali or an alkaline metal, such as sodium hydroxide, potassium hydroxide, sodium carbonate, potassium carbonate, sodium bicarbonate, potassium bicarbonate, and mixtures thereof. Preferably, the base is diisopropyl ethyl amine.

The solvent used during the reaction of a compound of Formula II with a compound of Formula III is selected from the group comprising water, ethers, esters, alcohols, hydrocarbons, halogenated hydrocarbons, and mixtures thereof. Examples of ether solvents include tetrahydrofuran and diisopropyl ether. Examples of ester solvents include ethyl acetate, butyl acetate, and isopropyl acetate. Examples of alcohol solvents include methanol, ethanol, and n-butanol. Examples of hydrocarbon solvents include hexane and heptane. An example of a halogenated hydrocarbon is dichloromethane. Preferably, the solvents used are tetrahydrofuran, ethyl acetate, water, or mixtures thereof.

In another embodiment of the present invention, the reaction of a compound of Formula II with a compound of Formula III is carried out in the presence of a phase transfer catalyst.

Examples of phase transfer catalysts include tetrabutylammonium iodide, tetrabutylammonium bromide, tetrabutylammonium fluoride, and mixtures thereof. Preferably, the phase transfer catalyst used is tetrabutylammonium iodide.

The reaction of a compound of Formula II with a compound of Formula III is carried out for about 10 hours to about 18 hours, for example, from about 12 hours to about 14 hours.

In another embodiment of the present invention, the reaction of a compound of Formula II with a compound of Formula III is carried out at a temperature of about 10° C. to about 100° C., for example, about 20° C. to about 80° C.

The compound of Formula IV, obtained by the reaction of a compound of Formula II with a compound of Formula III, may optionally be isolated by employing one or more techniques selected from the group consisting of filtration, decantation, extraction, distillation, evaporation, chromatography, precipitation, centrifugation, and recrystallization.

The compound of Formula V can be prepared by reacting a compound of Formula VIII with thiophosgene.

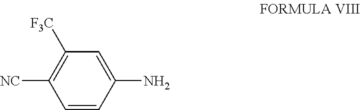 [SMILES: N#Cc1ccc(N)cc1C(F)(F)F]

The compound of Formula VIII can be prepared by the methods known in the art, for example, PCT Publication Nos. WO 2007/127010 and WO 2006/124118.

In another embodiment of the present invention, the reaction of a compound of Formula IV with a compound of Formula V is carried out in the presence of a solvent.

The solvent used during the reaction of a compound of Formula IV with a compound of Formula V is selected from the group consisting of water, dimethyl sulfoxide, esters, ethers, alcohols, hydrocarbons, halogenated hydrocarbons, or mixtures thereof. Examples of ester solvents include ethyl acetate, butyl acetate, and isopropyl acetate. Examples of alcohol solvents include methanol, ethanol, and n-butanol. Examples of hydrocarbon solvents include hexane and heptane. Examples of ether solvents include tetrahydrofuran and diisopropyl ether. An example of a halogenated hydrocarbon is dichloromethane. Preferably, a mixture of dimethyl sulfoxide, isopropyl acetate, methanol, and water is used as a solvent.

The reaction of a compound of Formula IV with a compound of Formula V is carried out for about 10 hours to about 18 hours, for example, from about 12 hours to about 14 hours.

In another embodiment of the present invention, the reaction of a compound of Formula IV with the compound of Formula V is carried out at a temperature of about 10° C. to about 100° C., for example, about 20° C. to about 80° C.

In another embodiment of the present invention, the compound of Formula I obtained by the reaction of a compound of Formula IV with a compound of Formula V is isolated by employing one or more techniques selected from the group consisting of filtration, decantation, extraction, distillation, evaporation, chromatography, precipitation, centrifugation, and recrystallization.

### Methods:

The IR spectrum was recorded using a PerkinElmer Spectrum One FT-IR spectrometer.

The Mass spectrum was recorded using an API 2000 LC/MS/MS system.

The NMR spectrum was recorded using a Bruker® Avance III 400 MHz NMR spectrometer.

While the present invention has been described in terms of its specific embodiments, certain modifications and equivalents will be apparent to those skilled in the art and are intended to be included within the scope of the present invention.

### EXAMPLES

### Example 1

### Process for the Preparation of 2-flouro-4-nitro benzoic acid (Formula VII)

### Method 1:

2-Flouro-4-nitro toluene (20 g) and sodium bromide (26.6 g) were added to de-ionized water (600 mL). Concentrated sulfuric acid (14 mL) was added to the reaction mixture and it was heated to 80° C. to 85° C. A solution of potassium bromate (21.6 g) in de-ionized water (400 mL) was added to the reaction mixture over 3 hours. The reaction mixture was stirred for 24 hours and cooled to 20° C. to 25° C. Ethyl acetate (300 mL) was added to the reaction mixture and it was stirred for 15 minutes. The layers obtained were separated. A solution of sodium hydroxide (10 g) in de-ionized water (300 mL) was added to the organic layer and it was stirred for 30 minutes. The layers obtained were separated and the aqueous layer was further washed with ethyl acetate (300 mL) to remove any undesired organic impurities. The aqueous layer was further acidified with concentrated hydrochloric acid (26 mL). Ethyl acetate (300 mL) was added to the aqueous layer. The ethyl acetate layer obtained was concentrated to obtain the title compound.

Yield: 15.4 g.

### Method 2:

2-Flouro-4-nitro toluene (20 g) was added to de-ionized water (600 mL) and heated to 50° C. Potassium permanganate (62 g) was added to the reaction mixture at 80° C. to 90° C. over 3 hours. The reaction mixture was stirred at 80° C. to 90° C. for 7 hours to 10 hours. The mixture was cooled to 20° C. to 25° C., filtered through celite, and washed with de-ionized water (600 mL) and ethyl acetate (600 mL). The reaction mixture was stirred for 30 minutes and the layers obtained were separated. The ethyl acetate layer was acidified using concentrated hydrochloric acid to bring the pH to 1-2, then stirred for 10 minutes. The layers obtained were separated and dichloromethane (400 mL) was added to the aqueous layer over 30 minutes. The layers obtained were further separated. The ethyl acetate and dichloromethane layers obtained were combined and concentrated to obtain the title compound.

Yield: 7.2 g.

### Example 2

### Process for the Preparation of N-methyl 2-flouro-4-nitro benzamide (Formula VI) from 2-flouro-4-nitro benzoic acid (Formula VII)

2-Flouro-4-nitro benzoic acid (Formula VII; 13 g) was added to dichloromethane (130 mL) and cooled to 0° C. to 5° C. A solution of methyl amine hydrochloride (9.48 g), hydroxybenzotriazole (9.49 g), and carbodiimide hydrochloride (17.5 g) in triethyl amine (34.3 mL) was added to the reaction mixture at 0° C. to 5° C. over 30 minutes. The reaction mixture was heated to 20° C. to 25° C. and stirred for 5 hours. De-ionized water (65 mL) was added to the reaction mixture. The layers obtained were separated and dichloromethane (65 mL) was added to the aqueous layer. Both of the organic layers were collected, combined, and washed with 1M hydrochloric acid solution (40 mL) for 10 minutes. The layers obtained were separated and the organic layer was washed with 5% (w/v) aqueous sodium bicarbonate solution for 15 minutes. The layers obtained were separated and the organic layer was further dried over sodium sulphate. The organic layer was filtered through a cotton plug and washed with dichloromethane (26 mL). The solution obtained was concentrated under reduced pressure to obtain the title compound.

Yield: 13.8 g.

### Example 3

### Process for the Preparation of N-methyl 2-flouro 4-amino benzamide (Formula II) from N-methyl 2-flouro 4-nitro benzamide (Formula VI)

N-Methyl 2-flouro-4-nitro benzamide (Formula VI; 13 g) was added to methanol (260 mL) followed by the addition of 10% (w/w) palladium/carbon (1.3 g) at 27° C. and hydrogen gas at 2 Kg/cm² to 2.5 Kg/cm² pressure for 2 hours to 4 hours. The reaction mixture was filtered through celite and washed with methanol (40 mL). The solution obtained was concentrated under reduced pressure at 30° C. to 35° C. for 1 hour to 2 hours to obtain the title compound.

Yield: 10.5 g.

### Example 4

### Process for the Preparation of ethyl N-[3-fluoro-4-(methylcarbamoyl)phenyl]-2-methylalaninate (Formula IV) from N-methyl 2-flouro 4-amino benzamide (Formula II

N-Methyl 2-flouro-4-amino benzamide (Formula II; 2 g) was added to ethyl 2-bromo isobutyrate (Formula III; 4 mL) and tetrahydrofuran (4 mL), followed by the addition of diisopropylethyl amine (2 mL) and tetrabutyl ammonium iodide (4 g). The reaction mixture was heated to 80° C. to 85° C. and a mixture of ethyl 2-bromo isobutyrate (2 mL) and diisopropyl ethyl amine (2 mL×4) was added to the reaction mixture over 8 hours. The reaction mixture was cooled to 20° C. to 25° C. followed by the addition of ethyl acetate (100 mL) and de-ionized water (100 mL). The reaction mixture was stirred for 30 minutes and the layers obtained were separated. The organic layer was separated and concentrated to obtain a residue which was further purified using silica gel column to obtain the title compound.

Yield: 1.5 g.

¹H NMR (400 MHz, CDCl₃), δ (in ppm): 7.9(t, j=9.04 Hz, 1H), 6.39(dd, j=2.32 Hz; 8.72 Hz, 1H), 6.2(dd, j=2.36 Hz; 15.16 Hz, 1H), 4.19(q, j=7.12 Hz, 2H), 2.99(d, 3H), 1.55(6H, s), 1.2(t, j=7.12 Hz, 3H).

Mass: [M+H]⁺=283.5; MS/MS: 283, 251.9, 224.1, 209.2, 178.1, 169.3, 152.1, 138, 112.1, 87.1, 58.1.

IR in KBr, (in cm⁻¹): 3348, 2980, 2936, 1730, 1624, 1607, 1551, 1517, 1407, 1384, 1343, 1315, 1299, 1268, 1223, 1179, 1146, 1108, 1019, 852, 832, 772, 755, 646, 619, 568, 527, 554, 466, 433.

### Example 5

### Process for the Preparation of 4-isothiocvanato 2-(triflouromethyl)benzonitrile (Formula V) from 4-amino 2-(triflouromethyl)benzonitrile (Formula VIII)

4-Amino-2-(triflouromethyl)-benzonitrile (Formula VIII; 20 g) was added to de-ionized water (100 mL) under stirring followed by the addition of thiophosgene (10 mL). The reaction mixture was stirred for 3 hours. Hexane (200 mL) was added to the reaction mixture and it was stirred for 30 minutes. The organic layer was concentrated to obtain the title compound.

Yield: 16.2 g.

### Example 6

### Process for the Preparation of Enzalutamide (Formula I)

Ethyl N-[3-fluoro-4-(methylcarbamoyl)-phenyl]-2-methylalaninate (Formula IV; 0.2 g) and 4-isothiocyanato 2-(triflouromethyl)-benzonitrile (Formula V; 0.33 g) were added to dimethyl sulfoxide (0.2 mL) and isopropyl acetate (0.4 mL) and heated to 90° C. to 95° C. The reaction mixture was cooled to 70° C. followed by the addition of methanol (0.4 mL). The reaction mixture was stirred for 2 hours. Isopropyl acetate (4 mL) was added to the reaction mixture followed by washing with water (4 mL). The organic layer was concentrated at 35° C. under vacuum to obtain an oily residue which was further purified using silica gel column to obtain the title compound.

Yield: 0.2 g

We claim:

1. A process for the preparation of enzalutamide of Formula I, 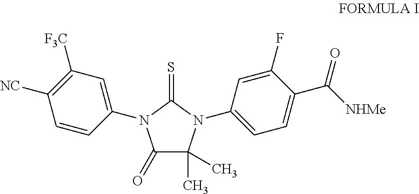 [SMILES: CNC(=O)c1ccc(N2C(=S)N(c3ccc(C#N)c(C(F)(F)F)c3)C(=O)C2(C)C)cc1F]
which comprises:
a) reacting a compound of Formula II 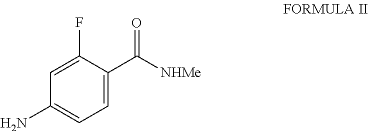 [SMILES: CNC(=O)c1ccc(N)cc1F]
with a compound of Formula III 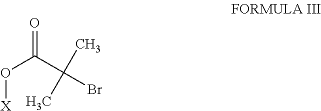
to prepare a compound of Formula IV; 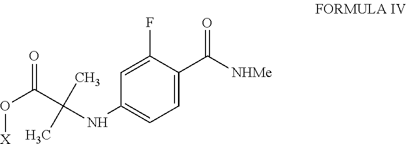
b) reacting the compound of Formula IV obtained in step a) with a compound of Formula V; and 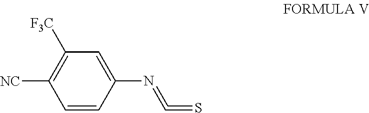 [SMILES: N#Cc1ccc(N=C=S)cc1C(F)(F)F]
c) isolating enzalutamide of Formula I from the reaction mixture of step b) wherein X is methyl, ethyl, or benzyl.

2. A process for the preparation of a compound of Formula IV, 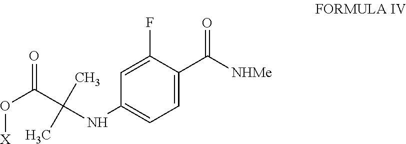
comprising reacting a compound of Formula II 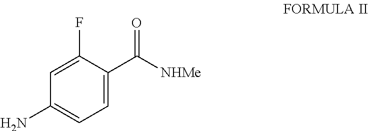 [SMILES: CNC(=O)c1ccc(N)cc1F]
with a compound of Formula III, 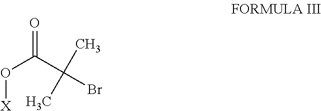
wherein X is methyl, ethyl, or benzyl.

3. A compound of Formula IV, wherein X is methyl, ethyl, or benzyl group.

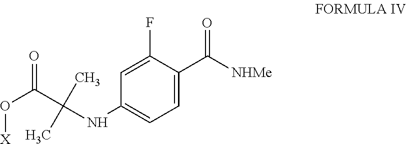

4. The process according to claim 1 or claim 2, wherein the reaction of the compound of Formula II with the compound of Formula III is carried out in the presence of a base and a solvent.

5. The process according to claim 4, wherein the base is an organic or an inorganic base.

6. The process according to claim 4, wherein the base is diisopropyl ethyl amine.

7. The process according to claim 4, wherein the solvent is selected from the group consisting of water, ethers, esters, alcohols, hydrocarbons, halogenated hydrocarbons, and mixtures thereof.

8. The process according to claim 4, wherein the solvent is selected from the group consisting of tetrahydrofuran, ethyl acetate, water, and mixtures thereof.

9. The process according to claim 1 or 2, wherein the reaction of the compound of Formula II with the compound of Formula III is carried out in the presence of a phase transfer catalyst.

10. The process according to claim 9, wherein the phase transfer catalyst is tetrabutylammonium iodide.

11. The process according to claim 1, wherein the reaction of the compound of Formula IV with the compound of Formula V is carried out in the presence of a solvent.

12. The process according to claim 11, wherein the solvent is selected from the group consisting of water, dimethyl sulfoxide, esters, ethers, alcohols, hydrocarbons, halogenated hydrocarbons, or mixtures thereof.

13. The process according to claim 11, wherein the solvent used is a mixture of dimethyl sulfoxide, isopropyl acetate, methanol, and water.

14. Use of a compound of Formula II or a compound of Formula IV for the preparation of enzalutamide.
